(19) United States
(12) Patent Application Publication
(10) Pub. No.: US 2026/0069277 A1
(43) Pub. Date: Mar. 12, 2026

(54) LEFT ATRIAL APPENDAGE CLOSURE DEVICE WITH MECHANICAL JOINING ASSEMBLY

(71) Applicant: BOSTON SCIENTIFIC SCIMED, INC., MAPLE GROVE, MN (US)

(72) Inventors: Christopher J Clark, St. Michael, MN (US); Ted William Pelzer, Otsego, MN (US); Neal Eidenschink, Wayzata, MN (US)

(73) Assignee: BOSTON SCIENTIFIC SCIMED, INC., MAPLE GROVE, MN (US)

(21) Appl. No.: 19/324,630

(22) Filed: Sep. 10, 2025

Related U.S. Application Data
(60) Provisional application No. 63/693,347, filed on Sep. 11, 2024.

(51) Int. Cl. A61B 17/12; A61B 17/00

(52) U.S. Cl. CPC A61B 17/12122; A61B 17/12172; A61B 2017/00477; A61B 2017/00867

(57) ABSTRACT

A left atrial appendage closure (LAAC) device includes a distal plate having a central aperture and a plurality of tine engagement regions that are circumferentially disposed about a periphery of the distal plate. An expandable frame extends between a proximal region and a distal region. The distal region includes a plurality of tines that each include an engagement end that is adapted to engage a corresponding one of the plurality of tine engagement regions such that the tines are prevented from moving within a plane extending through and parallel with the distal plate. A connection assembly is adapted to prevent the engagement ends of each of the tines from moving orthogonally to the plane extending through the distal plate.

### CROSS REFERENCE TO RELATED APPLICATIONS

This application claims the benefit of priority of U.S. Provisional Application No. 63/693,347 filed Sep. 11, 2024, the entire disclosure of which is hereby incorporated by reference.

### TECHNICAL FIELD

The disclosure relates generally to medical devices and more particularly to left atrial appendage closure devices.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device. The LAAC device includes a distal plate with a central aperture and a plurality of tine engagement regions that are circumferentially disposed about a periphery of the distal plate. The LAAC device includes an expandable frame that extends between a proximal region and a distal region, the distal region including a plurality of tines each having an engagement end that is adapted to engage a corresponding one of the plurality of tine engagement regions such that the tines are prevented from moving within a plane extending through and parallel with the distal plate. A connection assembly is adapted to prevent the engagement ends of each of the tines from moving orthogonally to the plane extending through the distal plate.

Alternatively or additionally, the connection assembly may include a first annular connection plate that is adapted to be positioned against a first side of the distal plate, an extension that extends from the first annular connection plate and is adapted to extend through the central aperture, and a second annular connection plate that is adapted to be positioned against the second side of the distal plate and including a connector aperture adapted to accommodate the extension therethrough.

Alternatively or additionally, the extension may be integrally formed with the first annular connection plate.

Alternatively or additionally, the second annular connection plate may be adapted to be secured to the extension in order to hold the connection assembly in place.

Alternatively or additionally, the connector aperture may be adapted to provide a compression fit with the extension.

Alternatively or additionally, the connector aperture and the extension may be adapted to provide a snap fit therebetween.

Alternatively or additionally, a portion of each of the plurality of tines may be at least substantially coplanar with the plurality of tine engagement regions within the distal plate.

Alternatively or additionally, each of the plurality of tine engagement regions and the corresponding engagement end of each of the plurality of tines may have complementary shapes that prevent each corresponding engagement end from being pulled out of the corresponding tine engagement region.

Alternatively or additionally, the plurality of tines may include nitinol.

Alternatively or additionally, the distal plate may include stainless steel.

Alternatively or additionally, the connection assembly may include stainless steel.

Alternatively or additionally, the LAAC device may further include an occlusive member extending over at least part of the proximal region of the expandable frame.

Alternatively or additionally, each of the engagement ends may include a frustoconical shape.

Alternatively or additionally, each of the plurality of engagement regions may include a corresponding frustoconical cutout.

Another example may be found in a left atrial appendage closure (LAAC) device. The LAAC device includes an expandable frame that extends between a proximal region and a distal region that includes a plurality of tines each having an engagement end and an occlusive covering that extends over at least part of the proximal region. The LAAC device includes a distal assembly including a distal plate, a plurality of tine engagement regions circumferentially arranged about the distal plate and each adapted to accommodate one of the engagement ends therein, a central aperture formed within the distal plate and disposed between the plurality of tine engagement regions, and a connection assembly that holds the engagement ends of each of the tines in position relative to the distal plate.

Alternatively or additionally, the connection assembly may include a first annular connection plate and an extension that is integrally formed with and extends orthogonally from the first annular connection plate and is adapted to extend through the central aperture. The connection assembly may include a second annular connection plate including a connector aperture adapted to accommodate the extension therethrough.

Alternatively or additionally, the first annular plate and the second annular plate may be adapted to sandwich the distal plate and the engagement ends of the tines therebetween.

Alternatively or additionally, the connector aperture may be adapted to be secured to the extension in order to hold the distal assembly together.

Alternatively or additionally, the connector aperture may be adapted to provide a mechanical lock with the extension.

Another example may be found in a left atrial appendage closure (LAAC) device. The LAAC device includes an expandable frame that extends between a proximal region and a distal region that includes a plurality of tines each having an engagement end. An occlusive covering extends over at least part of the proximal region. The LAAC device includes a distal assembly. The distal assembly includes a distal plate, a first annular connection plate that is disposed on a first side of the distal plate, and a second annular connection plate that is disposed on a second side of the distal plate. A plurality of tine engagement regions are circumferentially arranged about the distal plate and are each adapted to accommodate one of the engagement ends therein. The first annular connection plate is coupled to the second annular connection plate in order to hold the tines in position relative to the distal plate.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative LAAC device;

FIG. 2 is a distal end view of an illustrative expandable frame forming part of the illustrative LAAC device of FIG. 1;

FIG. 3 is an enlarged view of a portion of the illustrative expandable frame of FIG. 2;

FIG. 4 is a perspective view of an illustrative distal assembly that forms part of the illustrative LAAC device of FIG. 1;

FIG. 5 is an exploded perspective view of the illustrative distal assembly of FIG. 4;

FIG. 6 is a front view of a portion of the illustrative distal assembly of FIG. 4; and

FIG. 7 schematically shows several possible configurations for engagement ends of the illustrative distal assembly of FIG. 4.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A left atrial appendage closure (LAAC) device may be used to close off or otherwise occlude the volume within a left atrial appendage from the rest of the left atrium. Preventing blood flow in and out of the left atrial appendage can reduce the possibility of embolisms from forming within the left atrial appendage and reaching other parts of the body, including the brain. In some instances, a left atrial appendage closure (LAAC) device includes a distal plate with a central aperture and a plurality of tine engagement regions that are circumferentially disposed about a periphery of the distal plate. The LAAC device includes an expandable frame that extends between a proximal region and a distal region. The distal region includes a plurality of tines that each have an engagement end that is adapted to engage a corresponding one of the plurality of tine engagement regions such that the tines are prevented from moving within a plane extending through and parallel with the distal plate. A connection assembly is adapted to prevent the engagement ends of each of the tines from moving orthogonally to the plane extending through the distal plate.

In some cases, the connection assembly may include a first annular connection plate that is adapted to be positioned against a first side of the distal plate, an extension that extends from the first annular connection plate and is adapted to extend through the central aperture, and a second annular connection plate that is adapted to be positioned against the second side of the distal plate and includes a connector aperture that is adapted to accommodate the extension therethrough. The extension may be integrally formed with the first annular connection plate, for example. In some cases, the second annular connection plate may be adapted to be secured to the extension in order to hold the connection assembly in place. The connector aperture may be adapted to provide a compression fit with the extension. The connector aperture and the extension may be adapted to provide a snap fit therebetween.

In some cases, a portion of each of the plurality of tines may be at least substantially coplanar with the plurality of tine engagement regions within the distal plate. Substantially coplanar may be defined as the plurality of tines and the plurality of tine engagement regions being coplanar with one another within twenty percent or less, or within ten percent or less. In some cases, each of the plurality of tine engagement regions and the corresponding engagement end of each of the plurality of tines may have complementary shapes that prevent each corresponding engagement end from being pulled out of the corresponding tine engagement region. In some cases, the plurality of tines may include nitinol. In some cases, the distal plate may include stainless steel. In some cases, the connection assembly may include stainless steel. In some cases, the LAAC device may further include an occlusive member extending over at least part of the proximal region of the expandable frame. In some cases, each of the engagement ends may include a frustoconical shape and each of the plurality of engagement regions may include a corresponding frustoconical cutout.

In some instances, a left atrial appendage closure (LAAC) device includes an expandable frame extending between a proximal region and a distal region that includes a plurality of tines each having an engagement end. An occlusive covering extends over at least part of the proximal region. The LAAC device may include a distal assembly having a distal plate, a plurality of tine engagement regions that are circumferentially arranged about the distal plate and are each adapted to accommodate one of the engagement ends therein, a central aperture that is formed within the distal plate between the plurality of tine engagement regions, and a connection assembly that holds the engagement ends of each of the tines in position relative to the distal plate.

In some cases, the connection assembly may include a first annular connection plate, an extension that is integrally formed with and extending orthogonally from the first annular connection plate and is adapted to extend through the central aperture, and a second annular connection plate including a connector aperture that is adapted to accommodate the extension therethrough. In some cases, the first annular plate and the second annular plate may be adapted to sandwich the distal plate and the engagement ends of the tines therebetween. In some cases, the connector aperture may be adapted to be secured to the extension in order to hold the distal assembly together. As an example, the connector aperture may be adapted to provide a mechanical lock with the extension.

In some instances, a left atrial appendage closure (LAAC) device may include an expandable frame that extends between a proximal region and a distal region, with the distal region including a plurality of tines each having an engagement end. An occlusive covering extends over at least part of the proximal region. The LAAC device includes a distal assembly that includes a distal plate, a first annular connection plate that is disposed on a first side of the distal plate, and a second annular connection plate that is disposed on a second side of the distal plate. A plurality of tine engagement regions are circumferentially arranged about the distal plate and are each adapted to accommodate one of the engagement ends therein. The first annular connection plate is coupled to the second annular connection plate in order to hold the tines in position relative to the distal plate.

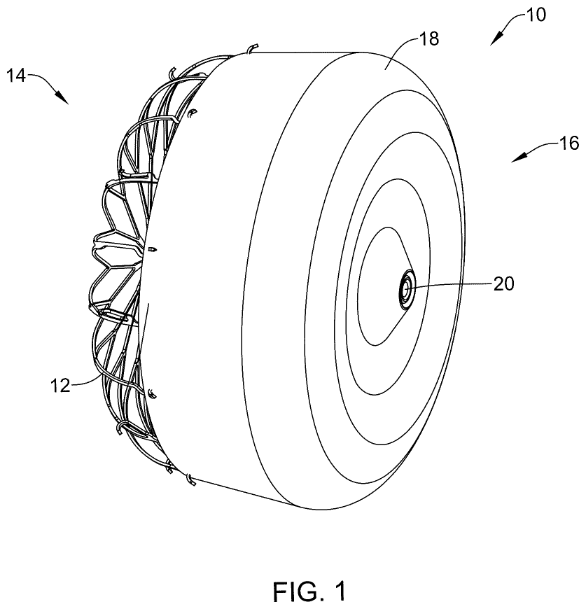

FIG. 1 is a side view of an illustrative LAAC device 10 that may be adapted to be implanted within a patient's LAA in order to occlude the patient's LAA and thus reduce or even eliminate the possibility for a blood clot within the LAA to exit the LAA and enter the patient's vasculature. An exemplary LAAC device 10 includes WATCHMAN FLX™ and WATCHMAN FLX™ Pro available from Boston Scientific.

The LAAC device 10 may include an expandable frame 12 that may be considered as including a distal region 14 and a proximal region 16. The expandable frame 12 may be movable between a collapsed configuration for delivery and an expanded configuration (as shown) when deployed. The LAAC device 10 may be adapted to be implanted within a patient's left atrial appendage in order to reduce or even eliminate blood flow into or out of the left atrial appendage. In some cases, an occlusive member 18 extends over at least a portion of the proximal region 16. The expandable frame 12 may define a hub 20 that allows for a delivery device (not shown) to be releasably secured to the LAAC device 10 for delivery and deployment. The hub 20 is adapted to allow a delivery device to be disconnected after the LAAC device 10 has been deployed, allowing the LAAC device 10 to remain behind in position within the patient's left atrial appendage.

In some cases, the occlusive member 18 may be permeable or impermeable to blood and/or other fluids, such as water. The occlusive member 18 may include a woven fabric/material or mesh, a non-woven fabric/material or mesh, a braided and/or knitted material, a fiber, a sheet-like material, a fabric, a mesh, a fabric mesh, a polymeric membrane, a metallic or polymeric mesh, a porous filter-like material, a covering, and/or other suitable construction, for example. In some cases, the occlusive member 14 may prevent thrombi (i.e. blood clots, etc.) from passing through the occlusive member 18 and out of the LAA into the blood stream. In some cases, the occlusive member 18 may promote endothelialization after implantation, thereby effectively removing the left atrial appendage from the patient's circulatory system.

The occlusive member 18 may be formed from a suitable material such as polyethylene terephthalate, polyester, nylon, acrylic materials, a polyolefin, and/or the like, combinations thereof, and/or other materials disclosed herein. In other instances, the occlusive material may include a metallic mesh formed from nickel-titanium alloy, stainless steel, titanium, other materials disclosed herein, combinations thereof, and/or the like.

In some examples, the expandable frame 12 may be integrally formed and/or cut from a unitary member. In some cases, the expandable frame 12 and may be integrally formed and/or cut from a unitary tubular member and subsequently formed and/or heat set to a desired shape in the expanded configuration. In some cases, the expandable frame 12 may be integrally formed and/or cut from a unitary flat member, and then rolled or formed into a tubular structure and subsequently formed and/or heat set to the desired shape in the expanded configuration. Some exemplary means and/or methods of making and/or forming the expandable frame 12 include laser cutting, machining, punching, stamping, electro discharge machining (EDM), chemical dissolution, etc. Other means and/or methods are also contemplated.

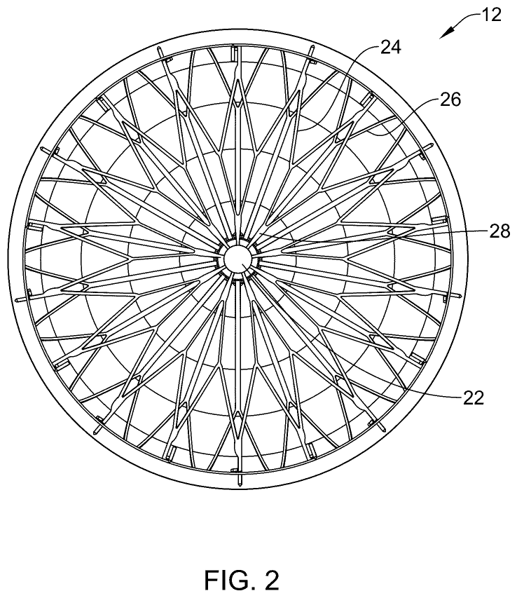

The expandable frame 12 may include a number of frame elements. As an example, the expandable frame 12 includes a number of frame elements 24 (only one is labeled in FIG. 2) that together form the distal region 14 of the expandable frame 12. The expandable frame 12 includes a number of frame elements 26 (only one is labeled in FIG. 2) that together form the proximal region 16 of the expandable frame 12. The frame elements 24, each of which may include one, two, three or more individual segments, terminate together in a distal strut 28 (only one is labeled). In some cases, each distal strut 28 may bifurcate into two individual segments, or may “tri-burcate” into three individual segments. Similarly, the frame elements 26, each of which may include one, two, three or more individual segments, terminate together at or near the hub 20. The expandable frame 12 may include any number of frame elements 24 and any number of frame elements 26. In some cases, each of the frame elements 24 may be considered as extending distally from a corresponding one of the frame elements 26. In some cases, each of the frame elements 26 may be considered as extending proximally from a corresponding one of the frame elements 24.

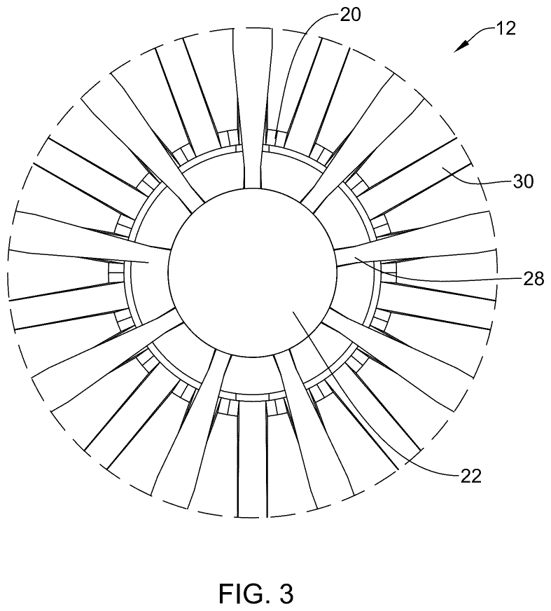

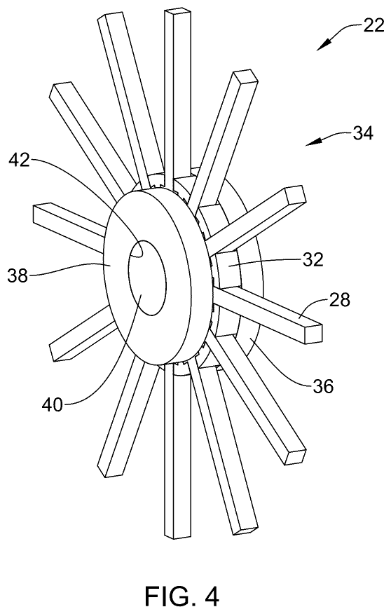

FIG. 2 is a distal end view of the expandable frame 12 and FIG. 3 is an enlarged distal end view of a central portion of the expandable frame 12. A connection assembly 22 may be seen in the middle of the distal end view, and will be described in greater detail with respect to FIGS. 4, 5 and 6. In some cases, the ends of the distal struts 28 may be joined together via the connection assembly 22. In some cases, the connection assembly 22 may enable the ends of the distal struts 28 to be joined together in a manner that does not require welding the ends of the distal struts 28 to any other structure (including the distal assembly 22). In some cases, the connection assembly 22 may enable a mechanical fixation of the ends of the distal struts 28. Also shown in FIG. 3 are the proximal struts 30 that serve to terminate the frame elements 26 and that are fixed to the hub 20.

It will be appreciated that the number of distal struts 28 may vary, depending on the exact design and size of the expandable frame 12. FIGS. 2 and 3 show an expandable frame 12 that includes a total of nine distal struts 28. In some cases, the expandable frame 12 may have fewer than nine distal struts 28. For example, the expandable frame 12 may have six, seven or eight distal struts 28. In some cases, the expandable frame 12 may have more than nine distal struts. For example, the expandable frame 12 may have ten, eleven, twelve or more distal struts 28. In some cases, the connection assembly 22 may be designed to accommodate any number of distal struts 28, depending on the overall design and construction of the expandable frame 12.

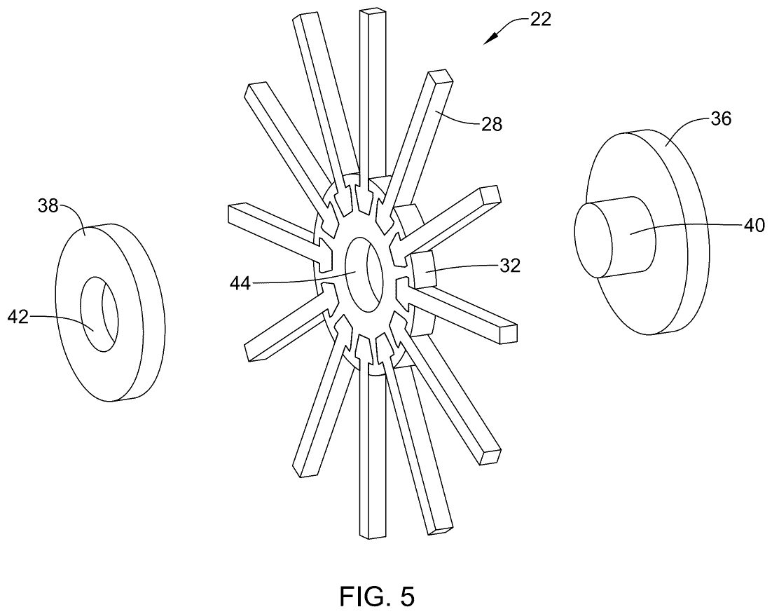

FIG. 4 is a perspective view of the illustrative connection assembly 22 shown in combination with the distal struts 28 while FIG. 5 is an exploded perspective view of the illustrative distal assembly 22. In FIGS. 4 and 5, the expandable frame 12 is shown as having a total of twelve distal struts 28. In some cases, the connection assembly 22 may be made to accommodate any number of distal struts 28. The distal assembly 22 includes a distal plate 32 and a connection assembly 34. In some cases, the distal plate 32 and the connection assembly 34 may, in combination, be considered as being a distal assembly, for example.

In some cases, the connection assembly 34 may include a first annular connection plate 36 and a second annular connection plate 38. As can be seen, the distal plate 32 may be sandwiched between the first annular connection plate 36 and the second annular connection plate 38. The first annular connection plate 36 may include an extension 40 that extends orthogonally from the first annular connection plate 36. In some cases, the extension 40 may be integrally formed with the first annular connection plate 36. In some cases, the extension 40 may be welded to the first annular connection plate 36. In some cases, the second annular connection plate 38 may include a connector aperture 42 that is adapted to accommodate the extension 40 therethrough. The distal plate 32 may include a central aperture 44 that allows the extension 40 to extend through the central aperture 44 and engage the connector aperture 42 within the second annular connection plate 38. In cases in which the connection assembly 22 is formed of a polymeric material, the second annular connection plate 38 may be secured to the extension 40 via heat-staking.

In some cases, the extension 40 may form a mechanical connection with the connector aperture 42. In some cases, the extension 40 may form a compressive fit within the connector aperture 42, requiring that the first annular connection plate 36 and the second annular connection plate 38 are pushed together with a particular force in order to urge the extension 40 into and through the connector aperture 42. In some cases, the extension 40 may fit easily through the connector aperture 42, and may be hammered or compressed in order to cause the extension 40 to mushroom in diameter and couple the first annular connector plate 36 to the second annular connector plate 38, much like a rivet. In some cases, the extension 40 may include a recessed area (not shown) that engages the connector aperture 42 once positioned. The extension 40 may be adhesively secured to the connector aperture 42. In some cases, the extension 40 may be welded to the second annular connection plate 38. In some cases, the extension 40 may be threaded, and may be adapted to threadedly engage a corresponding threaded surface formed on an inner surface of the connector aperture 42. In some cases, the extension 40 may form a snap fit within the connector aperture 42.

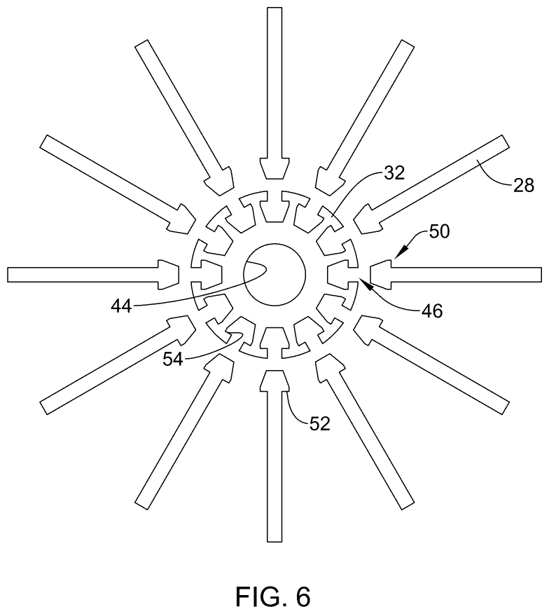

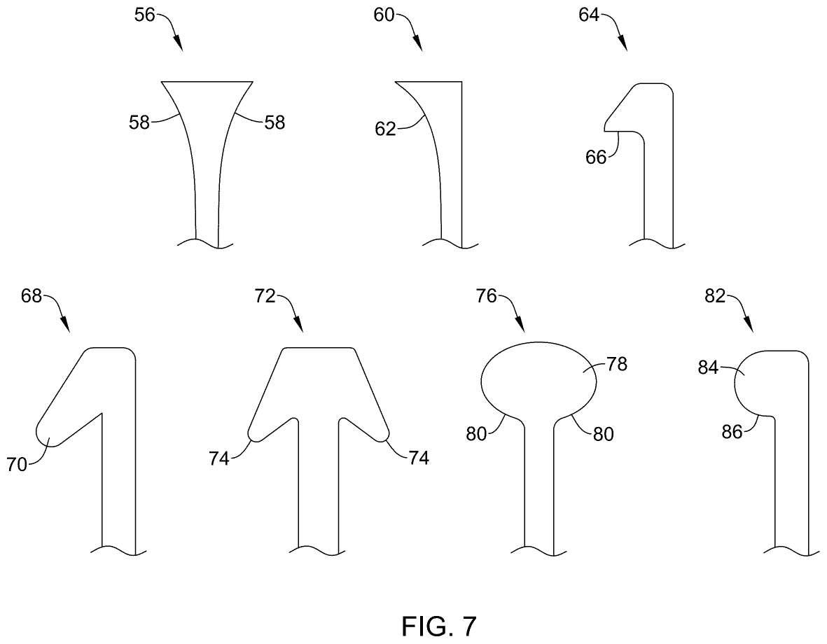

FIG. 6 is an exploded view showing the distal plate 32 in combination with the distal struts 28. The distal plate 32 includes a number of tine engagement regions 46 that are adapted to engage with corresponding engagement ends 50 of the distal struts 28. In this particular example, each of the tine engagement regions 46 include a frustoconical-shaped cutout and each of the corresponding engagement ends 50 have a corresponding frustoconical shape that is complementary to the frustoconical-shape cutouts forming each of the tine engagement regions 46. This is just an example, as the engagement ends 50 may have any of a variety of different shapes, and the tine engagement regions 46 may have any of a variety of complementarily-shaped cutouts to accommodate the engagement ends 50. As an example, the engagement ends 50 of the tines 28 may be T-shaped, and the tine engagement regions 46 may have a corresponding T-shaped cutout. In some cases, the engagement ends 50 and the tine engagement regions 46 may each have complementary geometries that prevent a distal strut 28 from being pulled in a radially-outwardly direction within a plane extending through and parallel with the distal plate 32. FIG. 7 provides additional examples of complementary shapes for the engagement ends 50 and the tine engagement regions 46.

As shown, each of the engagement ends 50 have a bearing surface 52 that will engage a corresponding bearing surface 54 within each tine engagement region 46. This means that once the engagement end 50 of each distal strut 28 is engaged within a corresponding tine engagement region 46 (as best seen in FIG. 5), each distal strut 28 is prevented from moving within the plate that extends through and is parallel with the distal plate 32, but of course would be free to move in a direction orthogonal to that plane. i.e., into the paper or out of the paper. In FIG. 6, this plane may be considered as being the paper on which FIG. 6 is printed.

The connection assembly 22 may be considered as being adapted to prevent movement of the distal struts 28 in a direction orthogonal to the plane extending through and parallel with the distal plate 32. Once assembled, the distal struts 28 are prevented from moving relative to the other distal struts 28 and relative to the distal plate 32 and the connection assembly 22. Hence, the connection assembly 22 provides a way to provide a mechanical securement of the distal struts 28 relative to each other without needing to weld the individual distal struts 28.

The connection assembly 22 may be assembled in either direction, meaning that the first annular connection plate 36 may be disposed within an interior of the expandable frame 12 while the second annular connection plate 38 may be disposed outside of the expandable frame 12, or in some cases, the first annular connection plate 36 may be disposed outside of the expandable frame 12 while the second annular connection plate 38 may be disposed within an interior of the expandable frame 12.

In some cases, the distal plate 32 may be formed via a stamping process. In some cases, the distal plate 32 may be formed via a machining process. The distal plate 32 may be laser-cut, for example. In some cases, the distal plate 32, the first annular connection plate 36 (including the extension 40) and the second annular connection plate 38 may be formed of any suitable metal, including stainless steel. The distal tines 28, as with the rest of the expandable frame 12, may be formed of nitinol. The distal plate 32 may be formed having a thickness that matches a thickness of the distal tines 28. In some cases, the distal plate 32 may be formed having a thickness that is greater than that of the distal tines 28, which may allow some movement orthogonal to the aforementioned plane. The connection assembly 22 would still prevent the distal tines 28 from pulling out of the distal plate 32, however.

As shown, the distal plate 32 has a symmetric profile, in which each of the tine engagement regions 46 are equally spaced around the circumference of the distal plate 32 and each of the tine engagement regions 46 are equally spaced in a radial direction relative to a periphery of the distal plate 32. The central aperture 44 is centered amongst all of the tine engagement regions 46. In some cases, the distal plate 32 may not have a symmetric profile. For example, some of the tine engagement regions 46 may not be equally spaced circumferentially and/or radially. Some of the distal tines 28 may be longer than others, for example.

In some cases, the distal tines 28 may be considered as being at least substantially coplanar, at least near the connection assembly 22. At least substantially coplanar may be defined as the distal tines 28 being coplanar with one another within twenty percent or less, or within ten percent or less. In some cases, this may mean that the distal tines 28 do not need to be bent or otherwise manipulated in order to be welded together. In some cases, the connection assembly 22 may simplify assembly of the expandable frame 12 by simplifying the process of securing the engagement ends 50 relative to each other.

FIG. 7 schematically shows several possible configurations for the engagement ends 50 of the distal tines 28 and the tine engagement regions 46. It will be appreciated that for each of the profiles shown, the profile represents a positive shape for the engagement ends 50 of the distal tines 28, and represents a corresponding cutout shape for the tine engagement regions 46 into which the engagement ends 50 fit. FIG. 7 includes a profile 56 that includes a curved surface 58 on either side of the profile 56. The curved surfaces 58 forming part of the engagement end 50 will engage the corresponding curved surface 58 forming part of the tine engagement region 46.

A profile 60 includes a single curved surface 62. The single curved surface 62 forming part of the engagement end 50 will engage the corresponding single curved surface 62 forming part of the tine engagement region 46. A profile 64 includes a flat surface 66. The flat surface 66 forming part of the engagement end 50 will engage the corresponding flat surface 66 forming part of the tine engagement region 46. A profile 68 includes a hook portion 70. The hook portion 70 forming part of the engagement end 50 will engage the corresponding hook portion 70 forming part of the tine engagement region 46. A profile 72 includes a hook portion 74 on either side. The hook portions 74 forming part of the engagement end 50 will engage the corresponding hook portions 74 forming part of the tine engagement region 46. A profile 76 includes a circular distal region 78 that includes curved surfaces 80 on either side. The curved surfaces 80 forming part of the engagement end 50 will engage the corresponding curved surfaces 80 forming part of the tine engagement region 46. A profile 82 includes a semi-circular distal region 84 that includes a curved surface 86. The curved surface 86 forming part of the engagement end 50 will engage the corresponding curved surface 86 forming part of the tine engagement region 46.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device, comprising:
a distal plate including:
a central aperture; and
a plurality of tine engagement regions circumferentially disposed about a periphery of the distal plate;
an expandable frame extending between a proximal region and a distal region, the distal region including a plurality of tines each having an engagement end that is adapted to engage a corresponding one of the plurality of tine engagement regions such that the tines are prevented from moving within a plane extending through and parallel with the distal plate; and
a connection assembly that is adapted to prevent the engagement ends of each of the tines from moving orthogonally to the plane extending through the distal plate.

2. The LAAC device of claim 1, wherein the connection assembly comprises:
a first annular connection plate adapted to be positioned against a first side of the distal plate;
an extension extending from the first annular connection plate and adapted to extend through the central aperture; and
a second annular connection plate adapted to be positioned against the second side of the distal plate and including a connector aperture adapted to accommodate the extension therethrough.

3. The LAAC device of claim 2, wherein the extension is integrally formed with the first annular connection plate.

4. The LAAC device of claim 2, wherein the second annular connection plate is adapted to be secured to the extension in order to hold the connection assembly in place.

5. The LAAC device of claim 2, wherein the connector aperture is adapted to provide a compression fit with the extension.

6. The LAAC device of claim 2, wherein the connector aperture and the extension are adapted to provide a snap fit therebetween.

7. The LAAC device of claim 1, wherein a portion of each of the plurality of tines are at least substantially coplanar with the plurality of tine engagement regions within the distal plate.

8. The LAAC device of claim 1, wherein each of the plurality of tine engagement regions and the corresponding engagement end of each of the plurality of tines have complementary shapes that prevent each corresponding engagement end from being pulled out of the corresponding tine engagement region.

9. The LAAC device of claim 1, wherein the plurality of tines comprise nitinol.

10. The LAAC device of claim 1, wherein the distal plate comprises stainless steel.

11. The LAAC device of claim 1, wherein the connection assembly comprises stainless steel.

12. The LAAC device of claim 1, further comprising an occlusive member extending over at least part of the proximal region of the expandable frame.

13. The LAAC device of claim 1, wherein each of the engagement ends include a frustoconical shape.

14. The LAAC device of claim 13, wherein each of the plurality of engagement regions include a corresponding frustoconical cutout.

15. A left atrial appendage closure (LAAC) device, comprising:
an expandable frame extending between a proximal region and a distal region, the distal region including a plurality of tines each having an engagement end;
an occlusive covering extending over at least part of the proximal region; and
a distal assembly including:
a distal plate;
a plurality of tine engagement regions circumferentially arranged about the distal plate and each adapted to accommodate one of the engagement ends therein;
a central aperture formed within the distal plate, the central aperture disposed between the plurality of tine engagement regions; and
a connection assembly that holds the engagement ends of each of the tines in position relative to the distal plate.

16. The LAAC device of claim 15, wherein the connection assembly comprises:
a first annular connection plate;
an extension integrally formed with and extending orthogonally from the first annular connection plate and adapted to extend through the central aperture; and
a second annular connection plate including a connector aperture adapted to accommodate the extension therethrough.

17. The LAAC device of claim 16, wherein the first annular plate and the second annular plate are adapted to sandwich the distal plate and the engagement ends of the tines therebetween.

18. The LAAC device of claim 16, wherein the connector aperture is adapted to be secured to the extension in order to hold the distal assembly together.

19. The LAAC device of claim 16, wherein the connector aperture is adapted to provide a mechanical lock with the extension.

20. A left atrial appendage closure (LAAC) device, comprising:
an expandable frame extending between a proximal region and a distal region, the distal region including a plurality of tines each having an engagement end;
an occlusive covering extending over at least part of the proximal region; and
a distal assembly including:
a distal plate;
a first annular connection plate disposed on a first side of the distal plate;
a second annular connection plate disposed on a second side of the distal plate;
a plurality of tine engagement regions circumferentially arranged about the distal plate and each adapted to accommodate one of the engagement ends therein;
wherein the first annular connection plate is coupled to the second annular connection plate in order to hold the tines in position relative to the distal plate.
